(19) United States
(12) Patent Application Publication
(10) Pub. No.: US 2023/0408843 A1
(43) Pub. Date: Dec. 21, 2023

(54) DIFFRACTIVE MULTIFOCAL OPHTHALMIC LENS WITH CHROMATIC ABERRATION CORRECTION

(71) Applicant: Alcon Inc., Fribourg (CH)

(72) Inventors: Myoung-Taek CHOI, Arlington, TX (US); Avni Ceyhun AKCAY, Mansfield, TX (US)

(21) Appl. No.: 18/337,645

(22) Filed: Jun. 20, 2023

Related U.S. Application Data
(60) Provisional application No. 63/366,663, filed on Jun. 20, 2022.

(51) Int. Cl. G02C 7/02; G02C 7/06

(52) U.S. Cl. CPC G02C 7/022; G02C 7/066; G02C 2202/20; G02C 2202/22

(57) ABSTRACT

Certain embodiments provide an intraocular lens (IOL) including a lens body having an anterior surface and a posterior surface, and a diffractive structure having a plurality of echelettes formed on at least one of the anterior surface or the posterior surface. A surface profile of the diffractive structure includes a base surface profile configured to diffract an incident light in one or more diffraction orders, and an achromatizing surface profile including increased step heights in the plurality of echelettes in relation to the base surface profile, and phase offsets between adjacent echelettes of the plurality of echelettes.

### CROSS-REFERENCE TO RELATED APPLICATIONS

This application claims priority to and benefit of U.S. Provisional Patent Application No. 63/366,663, filed Jun. 20, 2022. The aforementioned application is herein incorporated by reference in its entirety.

### BACKGROUND

The human eye in its simplest terms functions to provide vision by transmitting light through a clear outer portion called the cornea, and focusing the image by way of a lens onto a retina. The quality of the focused image depends on many factors including the size and shape of the eye, and the transparency of the cornea and lens. When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an intraocular lenses (IOLs).

IOLs are used for both refractive lens exchange and cataract surgery to replace the natural lens of the eyes and correct refractive errors. Among them are diffractive multifocal IOLs. However, in some instances, such diffractive multifocal IOLs may result in chromatic aberrations, which may affect visual acuity and contrast sensitivity.

### SUMMARY

Aspects of the present disclosure provide an intraocular lens (IOL) including a lens body having an anterior surface and a posterior surface, and a diffractive structure having a plurality of echelettes formed on at least one of the anterior surface or the posterior surface. A surface profile of the diffractive structure includes a base surface profile configured to diffract an incident light in one or more diffraction orders, and an achromatizing surface profile including increased step heights in the plurality of echelettes in relation to the base surface profile, and varied phase offsets by integer multiples of a design wavelength between adjacent echelettes of the plurality of echelettes.

Aspects of the present disclosure also provide an intraocular lens (IOL) including a lens body having an anterior surface and a posterior surface, and a diffractive structure having a plurality of echelettes formed on at least one of the anterior surface and the posterior surface. The diffractive structure is configured to provide a first focal point for distance vision, a second focal point for intermediate vision, and a third focal point for near vision for an incident light having a design wavelength, and a shift of the first focal point is less than 0.30 Diopter for an incident light having a wavelength that is different from the design wavelength by 50 nm.

Aspects of the present disclosure further provide an intraocular lens (IOL) including a lens body having an anterior surface and a posterior surface, and a diffractive structure having a plurality of echelettes formed on at least one of the anterior surface or the posterior surface. A surface profile of the diffractive structure includes a base surface profile configured to diffract an incident light in one or more diffraction orders, and an achromatizing surface profile comprising the plurality of echelettes with increased step heights in relation to the base surface profile, wherein at least one of the increased step heights is a non-integer multiple of a design wavelength.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is noted, however, that the appended drawings illustrate only some aspects of this disclosure and the disclosure may admit to other equally effective embodiments.

FIG. 1A depicts chromatic aberration of an exemplary refractive lens.

FIG. 1B depicts chromatic aberration of an exemplary diffractive lens.

FIG. 1C depicts chromatic aberration of an exemplary hybrid lens having a refractive lens portion and a diffractive lens portion.

FIG. 2A depicts a top view of an intraocular lens (IOL), according to certain embodiments.

FIG. 2B depicts a cross-sectional view of a portion of the IOL of FIG. 2A, according to certain embodiments.

FIG. 3 depicts a surface profile of a diffractive structure on an exemplary multifocal lens, according to certain embodiments.

FIG. 4A depicts a surface profile of a diffractive structure on an exemplary quadrafocal lens, according to certain embodiments.

FIG. 4B depicts diffraction efficiency of various diffraction orders of the exemplary quadrafocal lens of FIG. 4A, according to certain embodiments.

FIG. 4C depicts a surface profile of a diffractive structure on an exemplary quadrafocal lens, according to certain embodiments.

FIG. 4D depicts diffraction efficiency of various diffraction orders of the exemplary quadrafocal lens of FIG. 4C.

FIG. 4E depicts a surface profile of a diffractive structure on an exemplary quadrafocal lens, according to certain embodiments.

FIG. 4F depicts diffraction efficiency of various diffraction orders of the exemplary quadrafocal lens of FIG. 4E, according to certain embodiments.

FIG. 4G depicts a surface profile of a diffractive structure on an exemplary quadrafocal lens, according to certain embodiments.

FIG. 4H depicts diffraction efficiency of various diffraction orders of the exemplary quadrafocal lens of FIG. 4G, according to certain embodiments.

FIG. 4I depicts a surface profile of a diffractive structure on an exemplary quadrafocal lens, according to certain embodiments.

FIG. 4J depicts diffraction efficiency of various diffraction orders of the exemplary quadrafocal lens of FIG. 4I, according to certain embodiments.

FIG. 5A depicts a modulation transfer function (MTF) of the exemplary quadrafocal lens of FIG. 4A, according to certain embodiments.

FIG. 5B depicts a MTF of the exemplary quadrafocal lens of FIG. 4C, according to certain embodiments.

FIG. 5C depicts a MTF of the exemplary quadrafocal lens of FIG. 4E, according to certain embodiments.

FIG. 5D depicts a MTF of the exemplary quadrafocal lens of FIG. 4G, according to certain embodiments.

FIG. 6 depicts an example system for designing, configuring, and/or forming an IOL, according to certain embodiments.

FIG. 7 depicts example operations for forming an IOL, according to certain embodiments.

FIG. 8 depicts example steps of a method of achieving a shift in the diffraction order when forming a diffractive structure, according to certain embodiments.

FIG. 9 various example diffractive structure profiles with the same diffraction efficiency, according to certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The embodiments described herein provide a multifocal intraocular lens (IOL) having a diffractive structure designed for chromatic aberration correction, and methods and systems for fabricating the same. In certain embodiments, step heights of echelettes of the diffractive structure and phase offsets of the echelettes of the diffractive structure are configured such that diffraction orders that effectively correct chromatic aberration can be used for distance vision, intermediate vision, and near vision. For example, the step heights of each of the echelettes may be adjusted by an amount that is not limited to an integer multiple of a design wavelength, in order to shift diffraction orders that can be used for distance vision, intermediate vision, and near vision. In addition, phase offsets between adjacent echelettes may be configured such as to allow further chromatic aberration control without diffraction order shift and without diffraction efficiency change by varying integer multiple of a design wavelength. Thus, the IOLs according to the embodiments described herein provide increased design choices while chromatic aberration correction is improved.

A Diffractive Multifocal IOL with Chromatic Aberration Correction

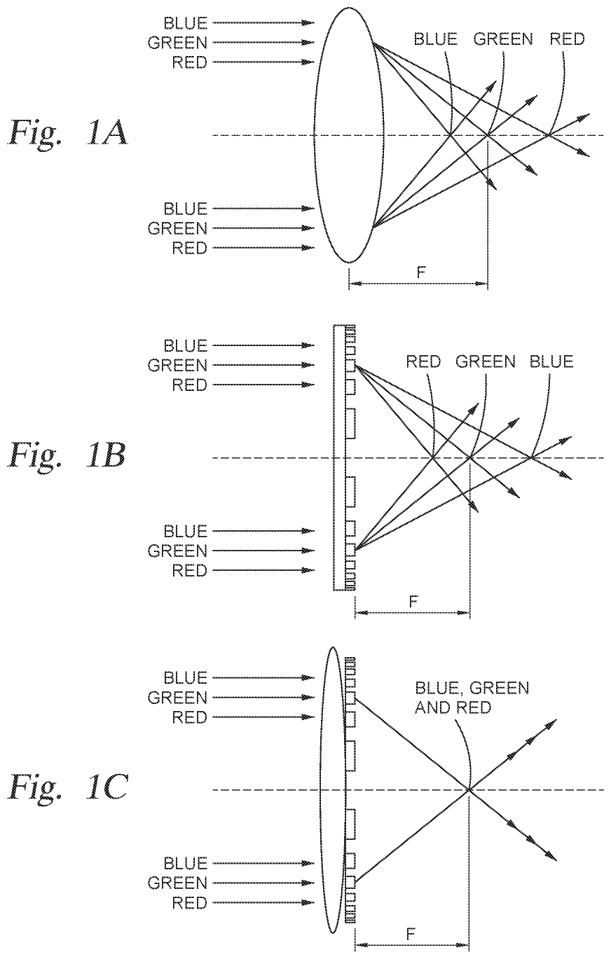

Chromatic aberration (i.e., a change in focal point versus wavelength) of a lens is due to either the dispersion properties (i.e., a change in refractive index versus wavelength) of the lens material or the lens structure. For a refractive lens, as in the example depicted in FIG. 1A, a longer wavelength focuses at a farther distance, since the refractive index of a typical lens material decreases at longer wavelengths. On the other hand, a diffractive lens, as in the example depicted in FIG. 1B, exhibits opposite chromatic aberration. A diffraction angle is proportional to wavelength, and thus a longer wavelength focuses at a shorter distance. Thus, in a lens having a diffractive lens portion and a refractive lens portion, the chromatic aberration due to the refractive lens portion can be compensated by the chromatic aberration due to the diffractive lens portion, and thus overall chromatic aberration of the lens may be corrected, as shown in FIG. 1C. Furthermore, the diffraction angle of the diffractive lens portion depends on diffraction orders. Thus, in the embodiments described herein, the diffractive structure on a diffractive lens portion is adjusted such that diffraction orders that effectively correct the overall chromatic aberration can be used.

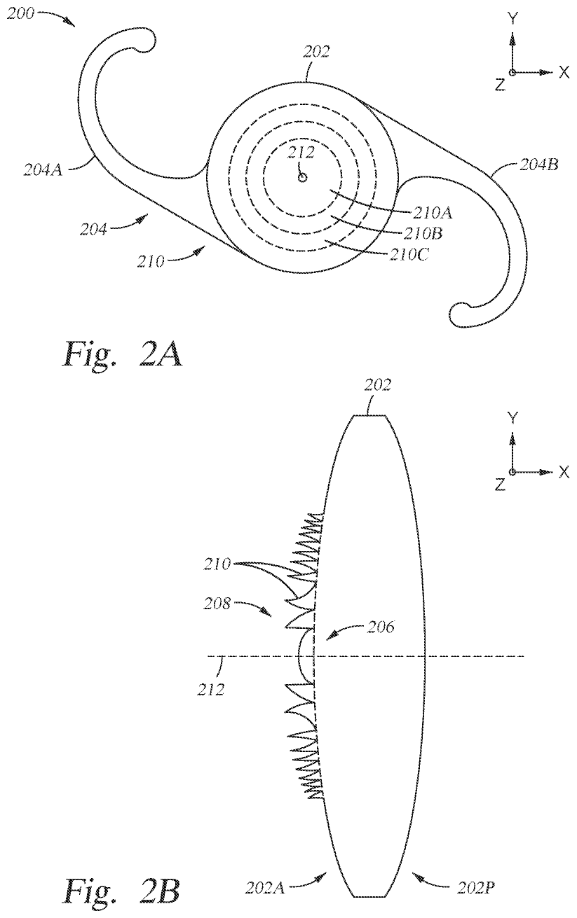

FIG. 2A depicts a top view of an intraocular lens (IOL) 200, according to certain embodiments. FIG. 2B depicts a side view of a cross-sectional view of the IOL 200. The IOL 200 includes a lens body 202 and a haptic portion 204 that is coupled to a peripheral, non-optic portion of the lens body 202.

The lens body 202 may be fabricated of biocompatible material, such as modified poly (methyl methacrylate) (PMMA), modified PMMA hydrogels, hydroxy-ethyl methacrylate (HEMA), PVA hydrogels, other silicone polymeric materials, and hydrophobic acrylic polymeric materials, for example, AcrySof® and Clareon®, available from Alcon, Inc., Fort Worth, Texas. The lens body 202 has a diameter ϕ of between about 4.5 mm and about 7.5 mm, for example, about 6.0 mm. It is noted that the shape and curvatures of the lens body 202 are shown for illustrative purposes only and that other shapes and curvatures are also within the scope of this disclosure. For example, the lens body 202 shown in FIG. 2B has a bi-convex shape. In other examples, the lens body 202 may have a plano-convex shape, a convexo-concave shape, or a plano-concave shape.

The haptic portion 204 includes hollow radially-extending struts (also referred to as “haptics”) 204A and 204B that are coupled (e.g., glued or welded) to the peripheral portion of the lens body 202 or molded along with a portion of the lens body 202, and thus extend outwardly from the lens body 202 to engage the perimeter wall of the capsular sac of the eye to maintain the lens body 202 in a desired position in the eye. The haptics 204A and 204B may be fabricated of biocompatible material, such as modified poly (methyl methacrylate) (PMMA), modified PMMA hydrogels, hydroxy-ethyl methacrylate (HEMA), PVA hydrogels, other silicone polymeric materials, and hydrophobic acrylic polymeric materials, for example, AcrySof® and Clareon®, available from Alcon, Inc., Fort Worth, Texas. The haptics 204A and 204B typically have radial-outward ends that define arcuate terminal portions. The terminal portions of the haptics 204A and 204B may be separated by a length L of between about 6 mm and about 22 mm, for example, about 13 mm. The haptics 104A and 104B have a particular length so that the terminal portions create a slight engagement pressure when in contact with the equatorial region of the capsular sac after being implanted. While FIG. 1A depicts one example configuration of the haptics 204A and 204B, any plate haptics or other types of haptics can be used.

The IOL 200 is a multifocal IOL (with multiple focal points, e.g., bifocal, trifocal, quadrafocal, and pentafocal) that is characterized by a base curvature 206 and a diffractive structure 208 formed on an anterior surface 202A of the lens body 202. The diffractive structure 208 diffracts an incident light into multiple diffraction orders and the light energy, power, or intensity of the incident light is divided into those multiple diffraction orders. Thus, a diffraction efficiency of each diffraction order is less than 100%. Although the diffractive structure 208 is shown only on the anterior surface 202A of the lens body 202 in FIG. 2B, the diffractive structure 208 may be formed on a posterior surface 202P of the lens body 202, or on both of the anterior surface 202A and the posterior surface 202P of the lens body 202.

The diffractive structure 208 includes multiple echelettes 210. A circular echelette 210A is centered at an optical axis 212 of the lens body 202 with a minimum radius. An annular echelette 210B adjacent to the circular echelette 210A is centered at the optical axis 212 of the lens body 202 with a radius larger than the minimum radius. An annular echelette 210C adjacent to the annular echelette 210B is centered at the optical axis 212 of the lens body 202 with a radius larger than the radius of the annular echelette 210B. In certain embodiments, the echelettes 210 include one or more annular echelettes (not numbered in FIG. 2A) surrounding the annular echelette 210C. As shown in FIG. 2B, step heights of the echelettes 210 may vary from one echelette to another echelette. In some other embodiments, the step heights of the echelettes 210 are constant across the surface of the lens body 202. Spacings (i.e., radial distances) between adjacent echelettes 210 may vary or be constant across the surface of the lens body 202. Step height of echelettes 210 are shown in units of Δn·λ, where Δn is a difference in the refractive indices of the lens body 202 and the surrounding media in which the lens body 202 is disposed.

In some embodiments, the diffractive structure 208 is used to provide a bifocal lens having two focal lengths for near and distance visions. A bifocal lens may utilize the first diffraction order for distance vision and the second diffraction order for near vision. In other embodiments, the diffractive structure 208 may provide a trifocal lens having three focal lengths for near, intermediate, and distance visions. A trifocal lens may utilize the zeroth diffraction order for distance vision, the first diffraction order for intermediate vision, and the second diffraction order for near vision. In other embodiments, the diffractive structure 208 is used to provide a quadrafocal lens. A quadrafocal lens may utilize the zeroth diffraction order for distance vision, the second diffraction order for intermediate vision, the third diffraction order for near vision, and the first diffraction order may be suppressed.

In certain embodiments described herein, to optimize the overall chromatic aberration correction, the diffractive structure 208 is adjusted to shift diffraction orders that are used for distance vision, intermediate vision, and near vision, by adjusting the step heights α of the echelettes 210 and phase offsets ϕ between adjacent echelettes 210.

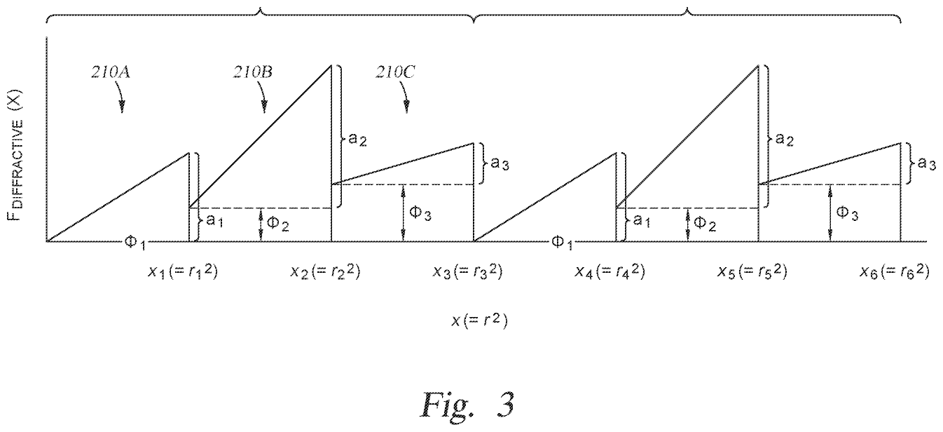

FIG. 3 depicts a surface profile F_{diffractive} (X) of a diffractive structure 208, showing height variation of the echelettes 210, on an exemplary multifocal lens. The surface profile F_{diffractive} (X) illustrates height variation of the echelettes 210 relative to the base curvature 206. As depicted, the circular echelette 210A has a radius r₁ (x₁=r₁²), a step height α₁, and a phase offset ϕ₁ relative to the base curvature 206. The annular echelette 210B surrounding the circular echelette 210A has a radius r₂ (x₂=r₂²), a step height α₂, and a phase offset ϕ₂ relative to the circular echelette 210A. The annular echelette 210C surrounding the annular echelette 210B has a radius r₃ (x₃=r₃²), a step height α₃, and a phase offset ϕ₃ relative to the annular echelette 210B.

In certain embodiments, as shown in FIG. 3, the surface profile F_{diffractive} (X) of the diffractive structure 208 for the three echelettes 210A, 210B, and 210C is repeated, such that the echelette having a radius r₄ (x₄=r₄²) has the same step height and the phase offset as the echelette 210A (i.e., a step height α₁ and a phase offset ϕ₁), the echelette having a radius r₅(x₅=r₅²) has the same step height and the phase offset as the annular echelette 210B (i.e., a step height α₂ and a phase offset ϕ₂), and the echelette having a radius r₅(x₅=r₅²) has the same step height and the phase offset as the echelette 210C (i.e., a step height α₃ and a phase offset ϕ₃).

The diffraction orders can be shifted by increasing all of the step heights α₁, α₂, α₃ and individually adjusting the phase offsets ϕ₁, ϕ₂, ϕ₃. The increase of all of the step heights can be by a non-integer multiple of wavelength λ. The phase offsets ϕ₁, ϕ₂, ϕ₃ can be increased or decreased by an integer multiple of wavelengths λ without affecting diffraction efficiencies or diffraction orders at the design wavelength, but affecting chromatic aberration. Thus, the phase offsets ϕ₁, ϕ₂, ϕ₃ can be adjusted to optimize the overall chromatic aberration correction, by increasing or decreasing the phase offsets by an integer multiple of wavelengths A.

### Examples

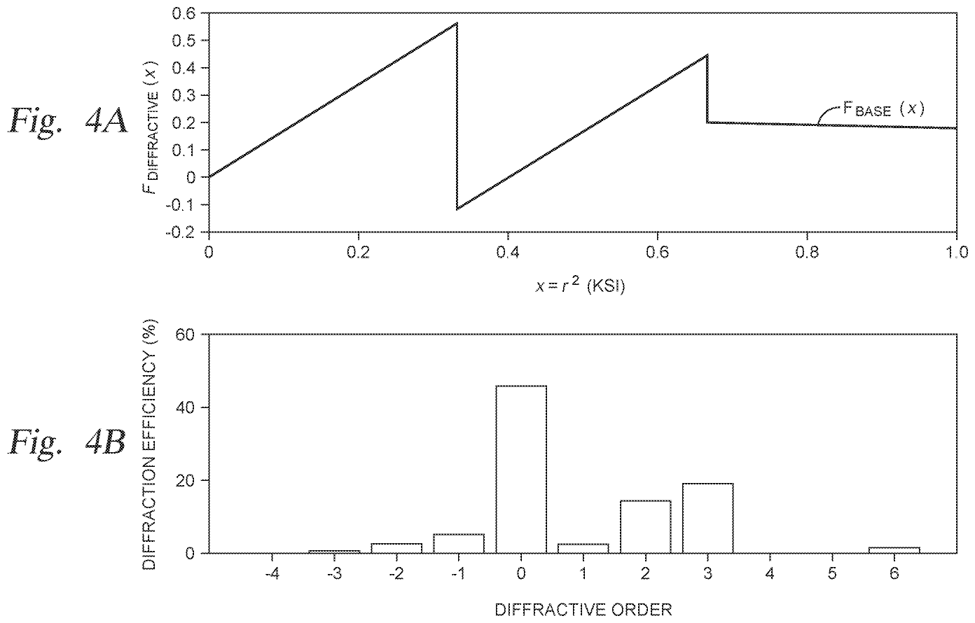

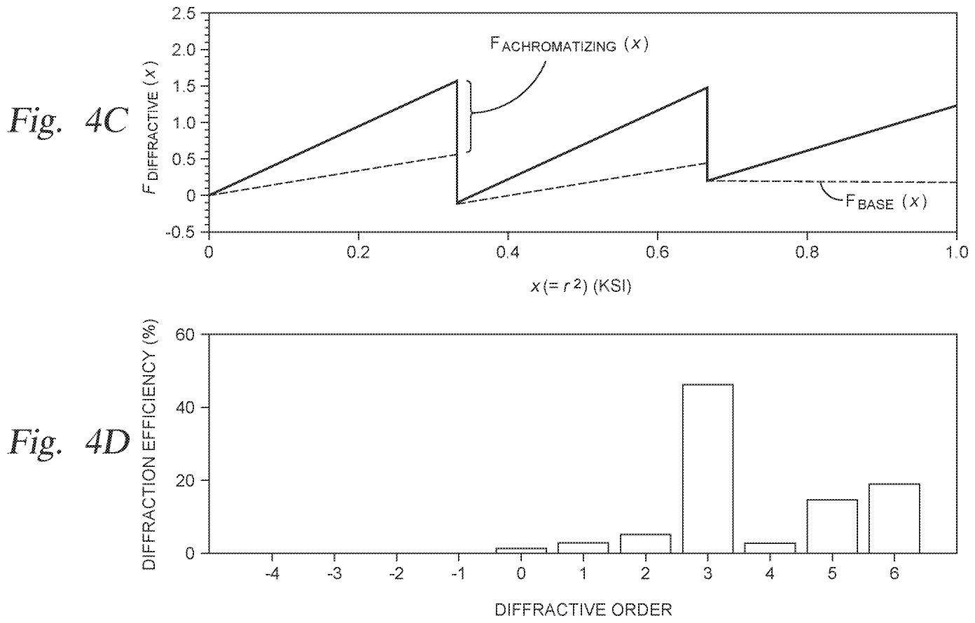

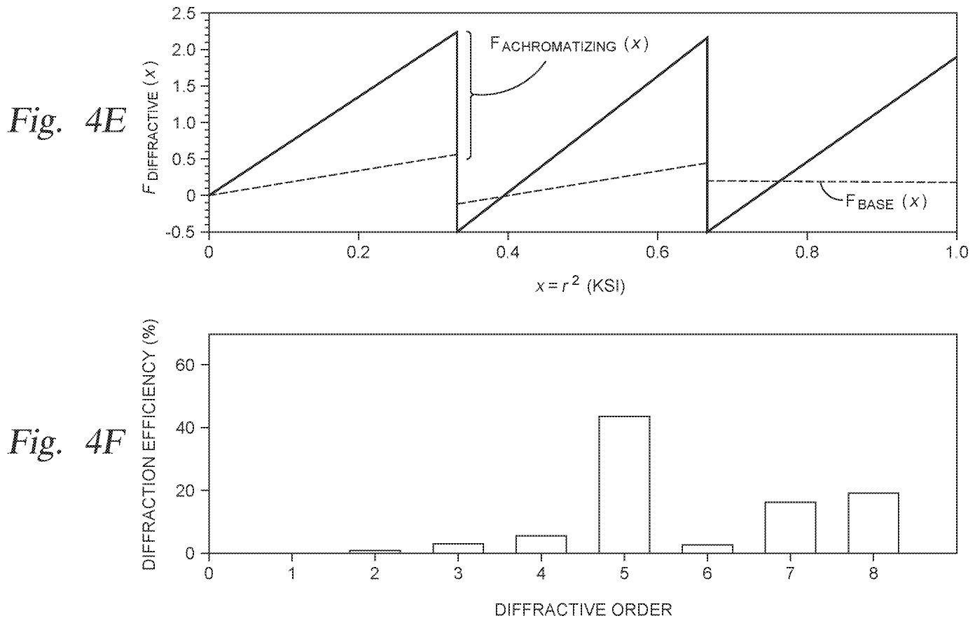

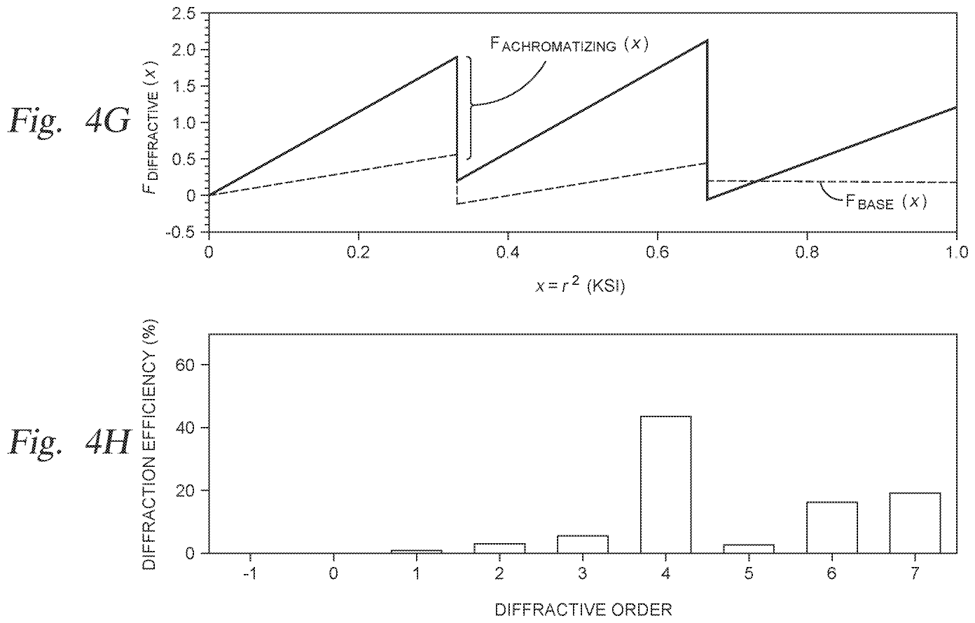

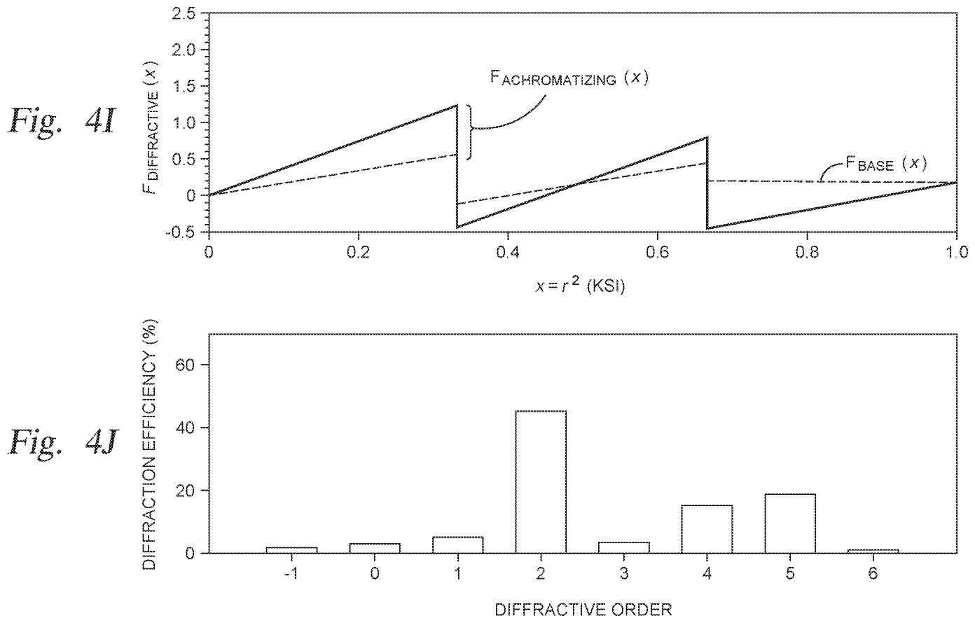

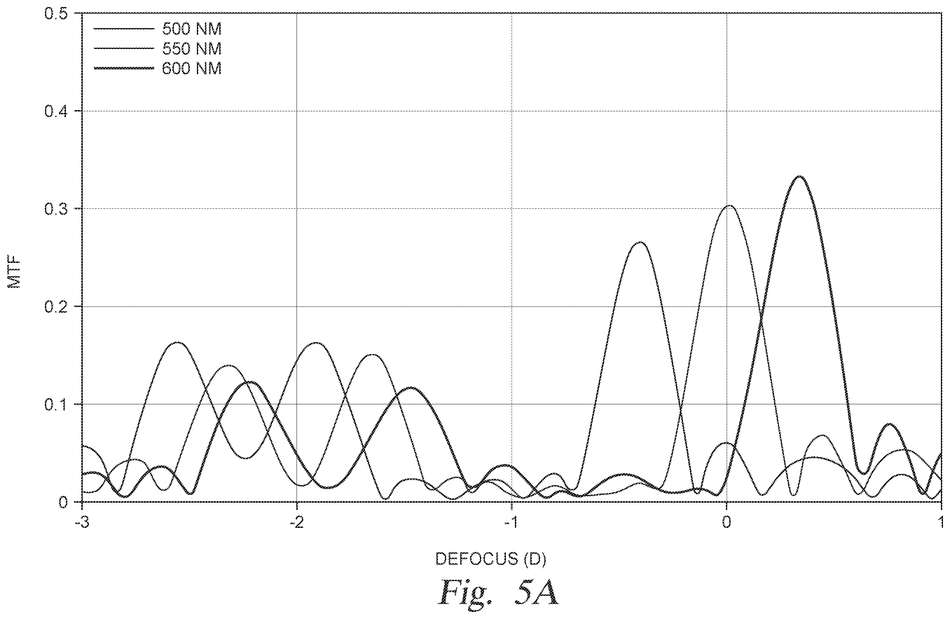

FIG. 4A depicts a surface profile F_{diffractive} (X) of a diffractive structure (e.g., diffractive structure 208) having echelettes (e.g., echelettes 210) on an exemplary quadrafocal lens (referred to as a “base design”). The surface profile F diffractive (X) of the base design is also referred to as a “base surface profile” and denoted as F_{base} (x). The surface profile F_{diffractive} (X) of the diffractive structure relative to its base curvature (e.g., base curvature 206) is shown in units of λ·Δn, where λ is a design wavelength (e.g., 550 nm) and Δn is a difference in the refractive indices of the lens and the surrounding media in which the lens is disposed. Here, x=r², where r is a radial distance from the optical axis of the lens (e.g., optical axis 212) normalized by period (r₃). FIG. 4B depicts diffraction efficiency at the design wavelength of various diffraction orders of the exemplary quadrafocal lens of the base design. In this example, the zeroth diffraction order may be used for distance vision, the second diffraction order may be used for intermediate vision, and the third diffraction order may be used for near vision. The first diffraction order may be suppressed.

FIG. 4C depicts a surface profile F_{diffractive} (X) of a diffractive structure (e.g., diffractive structure 208) having echelettes (e.g., echelettes 210) on an exemplary quadrafocal lens (referred to as a “shift+3 design”). In the shift+3 design, the surface profile F_{diffractive} (X) is the base surface profile F_{base} (x) with an achromatizing profile F_{achromatizing} (x) added thereon, in which the achromatizing profile F_{achromatizing} (x) includes an increase of all of the step heights a of the echelettes by one wavelength λ from the base surface profile F_{base} (x). The diffraction orders that provide high diffraction efficiencies, as depicted in FIG. 4D, are shifted by three as compared to those of the base design (depicted in FIG. 4B). In this example, the third diffraction order may be used for distance vision, the fifth diffraction order may be used for intermediate vision, and the sixth diffraction order may be used for near vision. The fourth diffraction order may be suppressed.

FIG. 4E depicts a surface profile F_{diffractive} (x) of a diffractive structure having echelettes on an exemplary quadrafocal lens (referred to as a “shift+5 design”). In the shift+5 design, the surface profile F_{diffractive} (X) is the base surface profile F_{base} (x) with an achromatizing profile F_{achromatizing} (x) added thereon, in which the achromatizing profile F achromatizing (x) includes an increase of all of the step heights α of the echelettes 210 by 5/3 wavelength λ from base surface profile F_{base} (x) and phase offsets ϕ₁=0, ϕ₂=−⅓,ϕ₃=−⅔ wavelengths λ. In an exemplary case, step heights of the echelettes 210 may include α₁=1.68, α₂=1.70, α₃=1.59 wavelengths from base surface profile F_{base} (x), and phase offsets ϕ₁=0, ϕ₂=−0.35, ϕ₃=−0.6 wavelengths, which are values that may be obtained using other numerical optimization techniques. The diffraction orders that provide high diffraction efficiencies, as depicted in FIG. 4F, are shifted by five as compared to those of the base design (depicted in FIG. 4B). In this example, the fifth diffraction order may be used for distance vision, the seventh diffraction order may be used for intermediate vision, and the eighth diffraction order may be used for near vision. The sixth diffraction order may be suppressed.

FIG. 4G depicts a surface profile F_{diffractive}(X) of a diffractive structure having echelettes on an exemplary quadrafocal lens (referred to as a “shift+4 design”). In the shift+4 design, the surface profile F_{diffractive} (X) is the base surface profile F_{base} (x) with an achromatizing profile F_{achromatizing} (x) added thereon, in which the achromatizing profile F_{achromatizing} (x) includes an increase of all of the step heights α of the echelettes 210 by 4/3 wavelength λ from base surface profile F_{base} (x) and phase offsets 0 1=0, =⅓, =−⅓ wavelengths λ. In an exemplary case, step heights of the echelettes 210 may include a₁=1.35, a 2=1.36, a 3=1.31 wavelengths from base surface profile F_{base} (x), and phase offsets ϕ₁=0, ϕ₂=0.3, ϕ₃=−0.3 wavelengths, which are values that may be obtained using other numerical optimization techniques. The diffraction orders that provide high diffraction efficiencies, as depicted in FIG. 4H, are shifted by four as compared to those of the base design (depicted in FIG. 4B). In this example, the fourth diffraction order may be used for distance vision, the sixth diffraction order may be used for intermediate vision, and the seventh diffraction order may be used for near vision. The fifth diffraction order may be suppressed.

FIG. 4I depicts a surface profile F_{diffractive} (X) of a diffractive structure having echelettes on an exemplary quadrafocal lens (referred to as a “shift+2 design”). In the shift+2 design, the surface profile F_{diffractive} (X) is the base surface profile F_{base} (x) with an achromatizing profile F_{achromatizing} (x) added thereon, in which the achromatizing profile F_{achromatizing} (x) includes an increase of all of the step heights a of the echelettes 210 by ⅔ wavelength π from base surface profile F_{base} (x) and phase offsets ϕ₁=0,ϕ₂=−⅓,ϕ₃=−⅔ wavelengths λ. The diffraction orders that provide high diffraction efficiencies, as depicted in FIG. 4J, are shifted by two as compared to those of the base design (depicted in FIG. 4B). In this example, the second diffraction order may be used for distance vision, the fourth diffraction order may be used for intermediate vision, and the fifth diffraction order may be used for near vision. The third diffraction order may be suppressed. In certain embodiments, the Shift+2 design maybe advantageous, particularly when used in conjunction with IOL material having lower material dispersion.

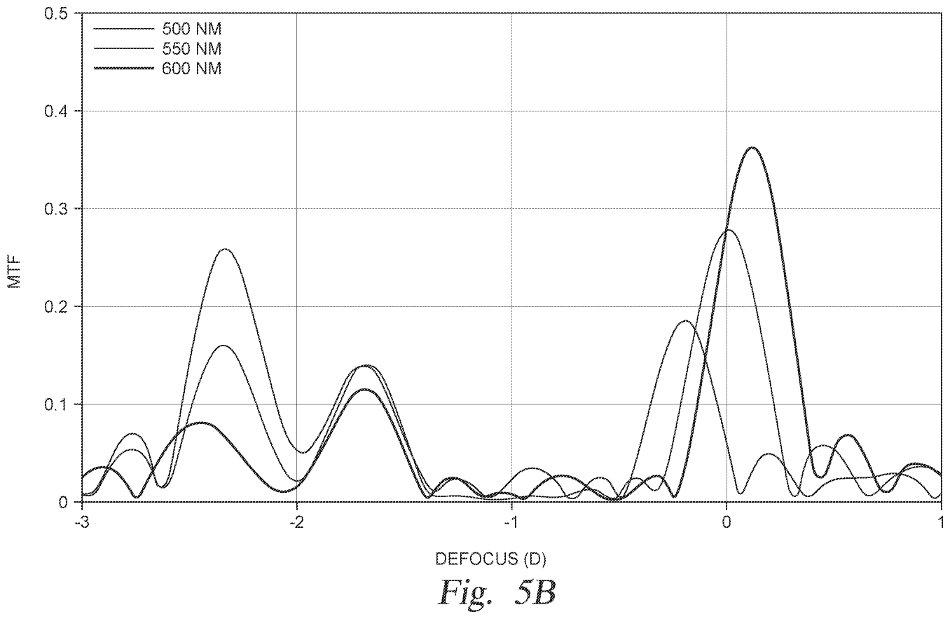

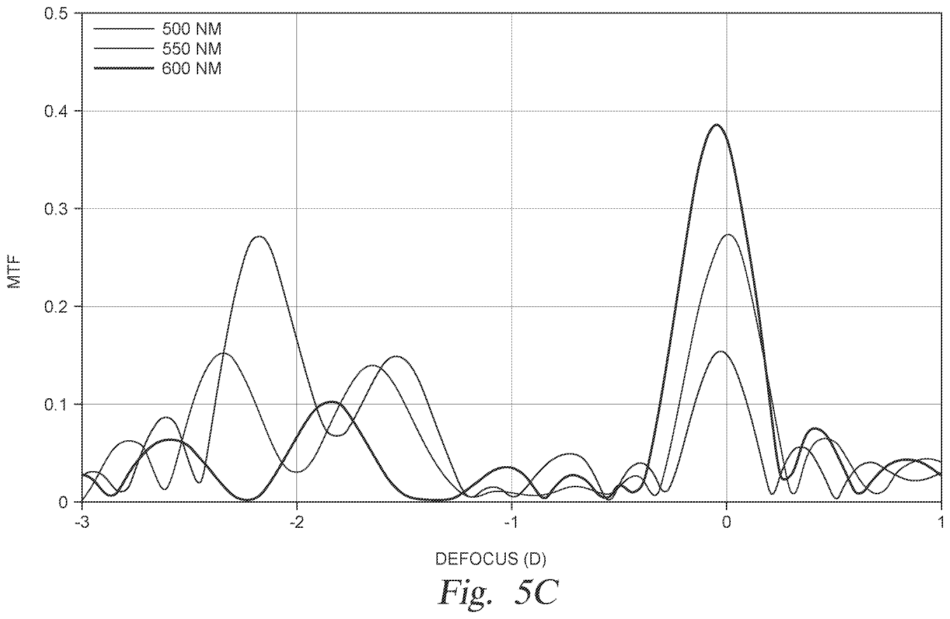

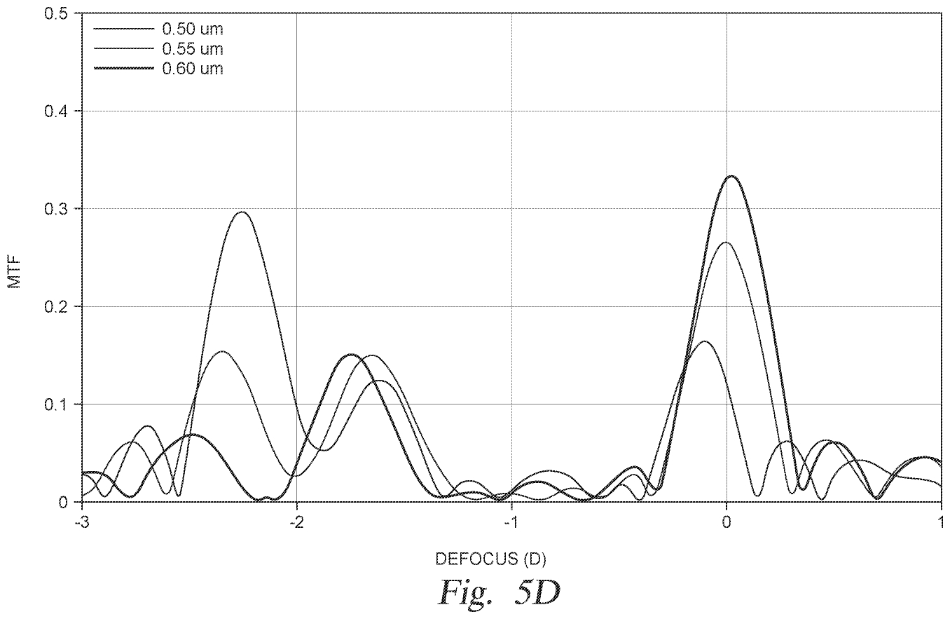

FIG. 5A depicts a modulation transfer function (MTF) of the exemplary quadrafocal lens of the base design shown in FIGS. 4A and 4B. The MTF was evaluated at a focus plane at 100 lm/mm (line pairs per millimeter) spatial resolution (also referred to as “spatial frequency”) using a 3-mm (photopic) aperture to determine a depth of focus (also referred to as “defocus”) for the lens. The peak positions at 0 Diopter, 1.5 Diopter, and 2.5 Diopter for the design wavelength (550 nm) correspond to the zeroth diffraction order for distance vision, the second diffraction order for intermediate vision, and the third diffraction order for near vision, respectively. As can be seen in FIG. 5A, this lens shows significant amount of chromatic aberration. All peak positions are significantly shifted for a shorter wavelength (500 nm) and a longer wavelength (600 nm). For example, the magnitude of shifting is 0.35 Diopter for a 600 nm wavelength and 0.45 for a 500 nm wavelength. In certain embodiments, the magnitude of shifting depends on the dispersion property of the lens material.

FIG. 5B depicts a MTF of the exemplary quadrafocal lens of the shift+3 design shown in FIGS. 4C and 4D, in which the achromatizing profile F_{achromatizing} (x) includes an increase of all of the step heights α of the echelettes 210 by one wavelength A from the from base surface profile F^{base}(x). As compared to the base design, shifting of the peak positions for wavelengths that are not a design wavelength (550 nm) is reduced. In particular, shifting of the peak at 1.5 Diopter (for intermediate vision) and the peak at 2.5 Diopter (for near vision), is significantly reduced. The peak is shifted by between about 0.3 Diopter and about 0.2 Diopter for a shorter wavelength (500 nm) and for a longer wavelength (600 nm), respectively. In certain embodiments, the magnitude of shift depends on the dispersion property of the lens material.

FIG. 5C depicts a MTF of the exemplary quadrafocal lens of the shift+5 design shown in FIGS. 4E and 4F, in which the achromatizing profile F_{achromatizing} (x) includes an increase of all of the step heights a of the echelettes 210 by 5/3 wavelength A from base surface profile F_{base}(x) and phase offsets φ₁=0, ϕ₂=−⅓, ϕ3=−2.3 wavelengths λ. As compared to the base design, shifting of the peak positions at 0 Diopter (for distance vision), at 1.5 Diopter (for intermediate vision), and at 2.5 Diopter (for near vision), is all significantly reduced. At distance, peak positions at three wavelengths are well aligned, thus negligible chromatic aberration is achieved. The shifting of the peak at 0 Diopter (for distance vision) due to chromatic aberration of the dispersion property of the lens material is reduced by using the diffraction orders that are shifted by 5 as compared to those of the base design. At intermediate/near, although significantly reduced compared to the base design, residual chromatic aberration may remain.

FIG. 5D depicts a MTF of the exemplary quadrafocal lens of the shift+4 design shown in FIGS. 4G and 4H, in which the achromatizing profile F_{achromatizing} (x) includes an increase of all of the step heights α of the echelettes 210 by 4/3 wavelength λ from base surface profile F_{base}(x) and phase offsets ϕ₁=0, ϕ₂=⅓, ϕ₃=−⅓ wavelengths λ. The amount of peak shifts are in-between the ‘ Shift+3’ and ‘ Shift+5’ designs. In the ‘ Shift+3’ design, distance is undercorrected, while in the ‘ Shift+5’ design, intermediate/near is overcorrected. On the other hand, in the Shift+4′ design, chromatic aberrations at all positions have a similar magnitude. The magnitude of shift depends on the dispersion property of the lens material.

### System for Designing an IOL

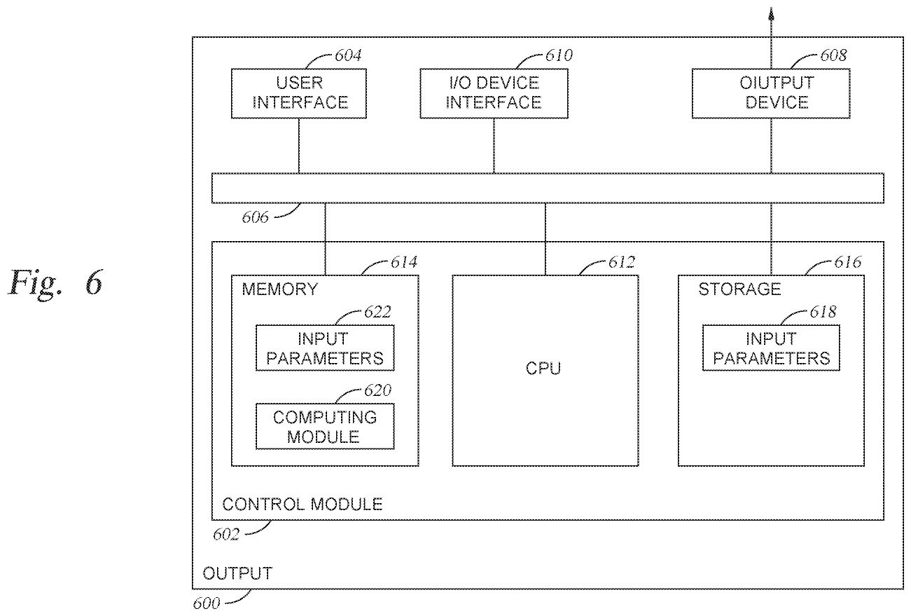

FIG. 6 depicts an exemplary system 600 for designing, configuring, and/or forming an IOL 200. As shown, the system 600 includes, without limitation, a control module 602, a user interface display 604, an interconnect 606, an output device 608, and at least one I/O device interface 610, which may allow for the connection of various I/O devices (e.g., keyboards, displays, mouse devices, pen input, etc.) to the system 600.

The control module 602 includes a central processing unit (CPU) 612, a memory 614, and a storage 616. The CPU 612 may retrieve and execute programming instructions stored in the memory 614. Similarly, the CPU 612 may retrieve and store application data residing in the memory 614. The interconnect 606 transmits programming instructions and application data, among CPU 612, the I/O device interface 610, the user interface display 604, the memory 614, the storage 616, output device 608, etc. The CPU 612 can represent a single CPU, multiple CPUs, a single CPU having multiple processing cores, and the like. Additionally, in certain embodiments, the memory 614 represents volatile memory, such as random access memory. Furthermore, in certain embodiments, the storage 616 may be non-volatile memory, such as a disk drive, solid state drive, or a collection of storage devices distributed across multiple storage systems.

As shown, the storage 616 includes input parameters 618. The input parameters 618 include a lens base power and a refractive index of a lens body. The memory 614 includes a computing module 620 for computing control parameters, such as step heights and phase offsets of echelettes of a diffractive structure. In addition, the memory 614 includes input parameters 622.

In certain embodiments, input parameters 622 correspond to input parameters 618 or at least a subset thereof. In certain embodiments, during the computation of the control parameters, the input parameters 622 are retrieved from the storage 616 and executed in the memory 614. In such an example, the computing module 620 comprises executable instructions for computing the control parameters, based on the input parameters 622. In certain other embodiments, input parameters 622 correspond to parameters received from a user through user interface display 604. In such embodiments, the computing module 620 comprises executable instructions for computing the control parameters, based on information received from the user interface display 604.

In certain embodiments, the computed control parameters, are output via the output device 608 to a lens manufacturing system that is configured to receive the control parameters and form a lens accordingly. In certain other embodiments, the system 600 itself is representative of at least a part of a lens manufacturing systems. In such embodiments, the control module 602 then causes hardware components (not shown) of system 600 to form the lens according to the control parameters. The details of a lens manufacturing system are known to one of ordinary skill in the art and are omitted here for brevity.

### Method for Forming an IOL

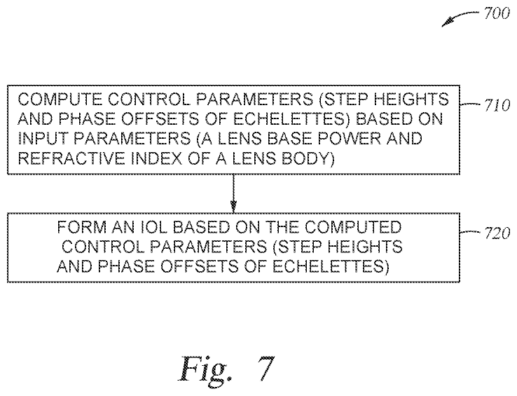

FIG. 7 depicts example operations 700 for forming an IOL (e.g., IOL 200). In some embodiments, the step 710 of operations 700 is performed by one system (e.g., the system 600) while step 720 is performed by a lens manufacturing system. In some other embodiments, both steps 710 and 720 are performed by a lens manufacturing system.

At step 710, control parameters, such as step heights and phase offsets of echelettes of a diffractive structure, are computed based on input parameters (e.g., a lens base power and a refractive index of the lens body). The computations performed at step 710 are based on one or more of the embodiments described herein. A variety of optimization techniques or algorithms may be used for selecting appropriate step heights and phase offsets of echelettes of a diffractive structure in order to optimize or maximize achromatization. For example, a method may be used to numerically minimize an error function for calculating the difference between the target and achieved diffraction efficiency, by varying design parameters.

As an alternative to using various optimization techniques for selecting appropriate step heights and phase offsets of echelettes of a diffractive structure in order to optimize or maximize achromatization, a method may be used for determining the step heights and phase offsets of echelettes of a diffractive structure for achieving a shift in the diffraction order.

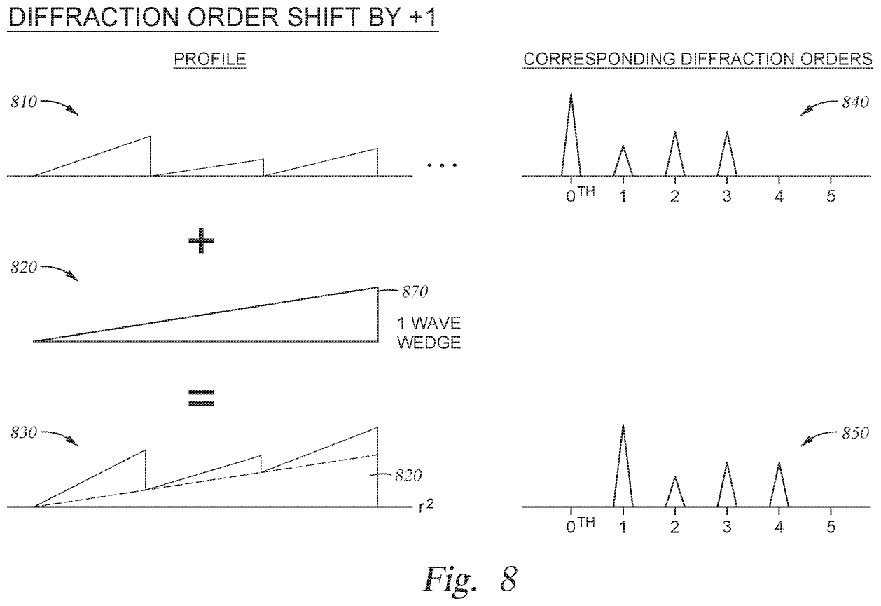

FIG. 8 illustrates an example method of determining appropriate step heights and phase offsets for a diffractive structure in order to shift the diffraction order by one (1) relative to a base profile. In particular, FIG. 8 shows a base profile 810 with a corresponding set of diffraction orders 840, centered around the 0^{th} order. In order to shift the diffraction order of the base profile 810 by one (1) (i.e., to achieve a “Shift+1” design), base profile 810 may be elevated in phase using a one-wave wedge 820 across the entire base profile 810, in the manner shown in FIG. 8, resulting in a diffractive structure 830 with a corresponding set of diffraction orders 850. As shown, there is a one-order shift (i.e., a shift from 0^{th} order to 1^{st} order) in diffraction orders 850 relative to diffraction orders 840. Note that, herein, a wedge is a triangular structure corresponding to the shape of a right triangle. In the embodiments of FIG. 8, the wedge 820 has a side 870 whose length defines a one-wave wedge. In other embodiments, as described below, wedges of various integer waves may be used. For example, a two-wave wedge (whose corresponding side has twice the length of side 870 of one-wave wedge 820), a three-wave wedge, or other multi-wave wedges may be used.

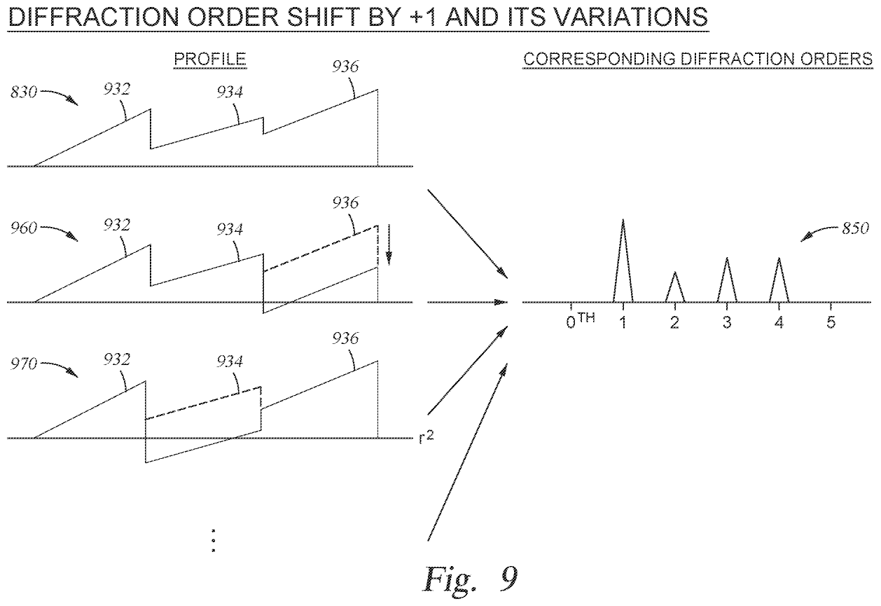

FIG. 9 illustrates various example diffractive structures, including diffractive structure 830 as well as other profiles or variations, with the same set of diffraction orders 850. The other variations of diffractive structure 830 are shown as diffractive structures 960 and 970. As illustrated, diffractive structures 830, 960, and 970 all have the same diffraction orders 850, thereby, all achieving the Shift+1 design with the same diffraction efficiency. Variations 960 and 970 may be formed by shifting (e.g., reducing) the phase of one or more echelettes 934 and 936 of diffractive structure 830 by an integer multiple of the design wavelength. For example, relative to diffractive structure 830, in diffractive structure 960, the phase of echelette 936 has been reduced across the entire echelette by one (1) wave. Note that diffractive structure 960 has the same diffractive efficiency, at the design wavelength, as diffractive structure 830. In another example, relative to diffractive structure 830, in diffractive structure 970, the phase of echelette 934 has been reduced across the entire echelette by one (1) wave. Note that diffractive structure 970 also has the same diffractive efficiency, at the design wavelength, as diffractive structures 830 and 960. Shifting the phase of echelettes 934 and 936 in other ways may also produce diffractive structures with the same Shift+1 design and diffractive efficiency.

Note that although FIGS. 8 and 9 only show the formation of diffractive structures with a Shift+1 design, diffractive structures with additional shifts in the diffraction order may be formed using similar techniques. For example, for the Shift+2 design, base profile 810 may be elevated in phase using a two-wave wedge across the entire base profile 810, to form a diffractive structure with a set of diffraction orders centered around the 2nd order. In such an example, other variations or profiles of the resulting diffractive structure may be generated by shifting down the phase of one or more echelettes of the resulting diffractive structure by one or more integer multiples of the design wavelength. Such variations may similarly have a set of diffraction orders centered around the second order and have the same diffraction efficiency as the diffractive structure produced as a result of elevating base profile 810 in phase using a two-wave wedge.

Referring back to FIG. 7, at step 720, an IOL (e.g., IOL 200) based on the computed control parameters, such as step heights and phase offsets of echelettes of a diffractive structure, is formed, using appropriate methods, systems, and devices typically used for manufacturing lenses, as known to one of ordinary skill in the art.

The embodiments described herein provide a multifocal intraocular lens (IOL) having a diffractive structure in which chromatic aberration is corrected. In the multifocal IOL according to the embodiments described herein, chromatic aberration of a refractive lens portion of the IOL due to the dispersion property of the lens material is compensated by chromatic aberration of a diffractive portion of the IOL, such that the overall chromatic aberration of the IOL is corrected. The overall chromatic aberration of the IOL can be optimized by adjusting step heights and phase offsets of the diffractive structure of the diffractive portion of the IOL. Such adjustments provide a wider variety of design choices to optimize chromatic aberration correction. For example, by allowing the step heights of the echelettes to be adjusted by an amount other than an integer multiple of a design wavelength, more precise control of chromatic dispersion correction may be achieved. In some instances, providing smaller step heights may also result in improved visual disturbance performance.

While the foregoing is directed to embodiments of the present disclosure, other and further embodiments of the disclosure may be devised without departing from the basic scope thereof, and the scope thereof is determined by the claims that follow.

1. An intraocular lens (IOL), comprising:
a lens body having an anterior surface and a posterior surface; and
a diffractive structure having a plurality of echelettes formed on at least one of the anterior surface or the posterior surface, wherein
a surface profile of the diffractive structure comprises:
a base surface profile configured to diffract an incident light in one or more diffraction orders; and
an achromatizing surface profile comprising:
increased step heights in the plurality of echelettes in relation to the base surface profile; and
phase offsets between adjacent echelettes of the plurality of echelettes.

2. The IOL of claim 1, wherein at least one of the increased step heights is a non-integer multiple of a design wavelength.

3. The IOL of claim 1, wherein the one or more diffraction orders provide distance vision, intermediate vision, and near vision.

4. The IOL of claim 3, wherein the achromatizing surface profile shifts the one or more diffraction orders to provide distance vision, intermediate vision, and near vision by two or four.

5. The IOL of claim 3, wherein the achromatizing surface profile shifts the one or more diffraction orders to provide distance vision, intermediate vision, and near vision by five.

6. The IOL of claim 1, wherein the lens body comprises hydrophobic acrylic polymeric material.

7. The IOL of claim 1, further comprising one or more haptics coupled to the lens body.

8. An intraocular lens (IOL), comprising:
a lens body having an anterior surface and a posterior surface; and
a diffractive structure having a plurality of echelettes formed on at least one of the anterior surface and the posterior surface, wherein
the diffractive structure is configured to provide a first focal point for distance vision, a second focal point for intermediate vision, and a third focal point for near vision for an incident light having a design wavelength, and
a shift of the first focal point is less than 0.3 Diopter for an incident light having a wavelength that is different from the design wavelength by between 40 and 70 nm.

9. The IOL of claim 8, wherein
a surface profile of the diffractive structure comprises:
a base surface profile configured to diffract an incident light in one or more diffraction orders; and
an achromatizing surface profile comprising:
increased step heights in the plurality of echelettes in relation to the base surface profile, and
phase offsets between adjacent echelettes of the plurality of echelettes.

10. The IOL of claim 9, wherein at least one of the increased step heights is a non-integer multiple of the design wavelength.

11. The IOL of claim 9, wherein the one or more diffraction orders provide distance vision, intermediate vision, and near vision.

12. The IOL of claim 11, wherein the achromatizing surface profile shifts the one or more diffraction orders to provide distance vision, intermediate vision, and near vision by two or four.

13. The IOL of claim 11, wherein the achromatizing surface profile shifts the one or more diffraction orders to provide distance vision, intermediate vision, and near vision by five.

14. The IOL of claim 9, wherein the lens body comprises hydrophobic acrylic polymeric material.

15. The IOL of claim 9, further comprising one or more haptics coupled to the lens body.

16. An intraocular lens (IOL), comprising:
a lens body having an anterior surface and a posterior surface; and
a diffractive structure having a plurality of echelettes formed on at least one of the anterior surface or the posterior surface, wherein
a surface profile of the diffractive structure comprises:
a base surface profile configured to diffract an incident light in one or more diffraction orders; and
an achromatizing surface profile comprising the plurality of echelettes with increased step heights in relation to the base surface profile, wherein at least one of the increased step heights is a non-integer multiple of a design wavelength.

17. The IOL of claim 16, wherein the one or more diffraction orders provide distance vision, intermediate vision, and near vision.

18. The IOL of claim 17, wherein the achromatizing surface profile shifts the one or more diffraction orders to provide distance vision, intermediate vision, and near vision by four.

19. The IOL of claim 17, wherein the achromatizing surface profile shifts the one or more diffraction orders to provide distance vision, intermediate vision, and near vision by five.

20. The IOL of claim 16, wherein the lens body comprises hydrophobic acrylic polymeric material.
